(19) United States
(12) Patent Application Publication
(10) Pub. No.: US 2026/0207461 A1
(43) Pub. Date: Jul. 23, 2026

(54) INGESTIBLE ORAL CARE COMPOSITIONS AND USES THEREOF

(71) Applicant: EDICCARE INC., Richmond (CA)

(72) Inventors: Jin Ying ZHAO, Richmond (CA); Xu Dong DONG, Richmond (CA)

(73) Assignee: EDICCARE INC., Richmond, BC (CA)

(21) Appl. No.: 19/143,074

(22) PCT Filed: Dec. 29, 2023
(86) PCT No.: PCT/CA2023/051776

§ 371 Date: Jun. 25, 2025

Related U.S. Application Data
(60) Provisional application No. 63/436,529, filed on Dec. 31, 2022.

(51) Int. Cl. A61K 8/64; A61K 8/37; A61K 8/67; A61K 8/73; A61K 8/92; A61Q 11/00

(52) U.S. Cl. CPC A61K 8/64; A61K 8/375; A61K 8/673; A61K 8/676; A61K 8/733; A61K 8/737; A61K 8/922; A61Q 11/00; A61K 2800/92

(57) ABSTRACT

The present disclosure relates to ingestible oral care compositions and uses thereof, for example for controlling dental plaque and/or gum inflammation in a subject. For example, the ingestible oral care compositions can comprise: protein, lipids, polysaccharides and/or derivatives thereof, and optionally an active, in a water-based orally acceptable vehicle.

### CROSS-REFERENCE TO RELATED APPLICATIONS

The present disclosure claims the benefit of priority from U.S. provisional application No. 63/436,529 filed on Dec. 31, 2022, the contents of which are incorporated herein by reference in their entirety.

### FIELD

The present disclosure relates to ingestible oral care compositions as well as uses thereof, for example for controlling dental plaque and/or gum inflammation in a subject.

### BACKGROUND

Dental plaque is a sticky film that coats teeth and contains bacteria. It is associated with common dental diseases, especially tooth decay and gingival diseases. Recent studies have also shown that dental plaque infection could contribute to systemic inflammation. Dental plaque is a biofilm composed of micro-organisms distributed in an intercellular matrix. The matrix primarily is made of the following organic constituents: polysaccharides, proteins, glycoprotein, and lipids; inorganic constituents: calcium, phosphorus, sodium, potassium, and fluoride, among which exopolysaccharides produced by the bacteria make up 50-95% of the dry weight of the plaque biofilm. These constituents contribute to the structural integrity of the biofilm and the attachment of the biofilm to the tooth surfaces.

About 50% of the adult population is affected by gingivitis globally. Gingivitis and periodontitis are a continuous disease of the same inflammation, and also, not all patients with gingivitis will progress to periodontitis. Management of gingivitis is both a primary prevention strategy for periodontitis and a secondary prevention strategy for recurrent periodontitis. The most important risk factor for gingivitis and periodontitis is the accumulation of a plaque biofilm at and below the gingival margin, within which dysbiosis develops and is associated with an inappropriate and destructive host inflammatory immune response. Therefore, oral care products that can be applied locally to a gingival tissue to control gingival inflammation, prevent gingivitis and/or prevent gingivitis from progressing to periodontitis would be desirable for managing gum diseases.

Daily brushing, flossing and use of antimicrobial mouth rinses are the commonly recommended measures to control plaque accumulation. Although these measures can provide temporary removal and/or control of the biofilm, the biofilm cannot be completely eliminated. Additionally, frequent mechanical interventions could lead to detrimental effects on the soft tissues surrounding the teeth if not controlled well and contribute to gum recession, especially in aging populations. Although dental plaque can be controlled by these measures, because of the dynamic interaction between the host and the microbiome community, the diet and/or stress of modern life, there is a need for non invasive adjunctive measures to help soothe the soft tissues after mechanical interferences, maintain the homeostasis of the oral microbiota and prevent biofilm induced virulence and/or inflammation. An oral care product that is safe and convenient for daily use and provides longer and more efficacious protection of the gingiva and teeth from biofilm proliferation and dysbiosis would complement the current oral care regimen and provide improved maintenance and potentially enhancement of oral health and potentially systemic health which is related to oral health conditions. However, the moist environment and dynamic function of the mouth can pose significant challenges for leave-in oral care products. Another challenge of ingestible oral care products is the potential of plaque regrowth (see, for example: Rubido et al., In vivo antiplaque effect of three edible toothpastes Med Oral Patol Oral Cir Bucal. 2014 Jan. 1; 19(1):e88-92).

### SUMMARY

Briefly stated, the present disclosure includes methods and compositions for an application to teeth and gum surfaces that lasts for an extended period of time on the contact surfaces and is safe to be ingested after application. Surprisingly, the highly biocompatible ingestible oral compositions of the disclosure controlled plaque regrowth and accumulation in a non-invasive manner without mechanical force or anti-microbial chemicals.

When applied on dental surfaces, exemplary ingestible oral care compositions of the present disclosure attached to the existing biofilm, altered the cohesiveness of the EPM and reduced the mechanical stability of the matrix, while at the same time, acted as a physical barrier to prevent further bacteria attachment to the existing biofilm. While not wishing to be limited by theory, this highly biocompatible complex seems to be acting like a camouflage of the oral biofilm, which attenuates biofilm growth and preserves the biofilm at levels compatible to oral health through destabilizing the existing biofilm and distracting its growth.

The present disclosure includes an ingestible oral care composition, comprising: protein; lipids; polysaccharides and/or derivatives thereof; and optionally an active, in a water-based orally acceptable vehicle.

In an embodiment, the ingestible oral care composition further comprises a pH neutralizing agent. In an embodiment, the ingestible oral care composition comprises the active.

The present disclosure also includes a use of an ingestible oral care composition as described herein for controlling gum inflammation in a subject. The present disclosure also includes a use of an ingestible oral care composition as described herein, which comprises an active, for controlling respiratory diseases through mouth and throat in a subject. The present disclosure also includes a use of an ingestible oral care composition as described herein for preventing systemic diseases related to periodontal inflammation in a subject. The present disclosure also includes a use of an ingestible oral care composition as described herein for non-invasively destabilizing plaque, preventing regrowth of plaque and/or reducing plaque on an oral surface of a subject. The present disclosure also includes a use of an ingestible oral care composition as described herein, which comprises an active, for delivering the active to a subject in need thereof.

Certain embodiments of the present disclosure include, but are not limited to: a) brushing the product all over the tooth and gum surfaces using a soft toothbrush and leaving it to slowly disperse into oral cavity, preferably at night after general tooth cleaning with tooth paste and dental floss, wherein the oral care product performs as a leave in conditioning agent after tooth cleaning, an easy and convenient complement to the current oral care regimen; b) brushing or spreading the product by hand on specific targeted tooth and gum surfaces i.e. areas with dental concerns including pain, bleeding, sensitivity, swollen, gingiva inflammation, gum recession, and etc. whenever necessary, with or without the help of a clean dental floss to push the product further into the base of the teeth or the periodontal pockets for maximum effect; c) applying the product as a medication to target spots on tooth and gum surfaces, or to the base of specific teeth or to specific periodontal pockets directly by hand, or with brush, or with the help of a clean dental floss, or with a specific dental product application tool such as a curve tipped syringe for therapeutic purposes, in this feature, therapeutic agents suitable for topical applications are carried in the product; d) including health promoting components or drugs that provide additional benefits for systemic health or particularly respiratory health through regular use of the product, which is enabled by the prolonged releases and ingestions after application. The aforementioned extended period of time of contact on the tooth and gum surfaces enables longer local delivery time and creates an opportunity for more efficient delivery of health promoting ingredients or medications and safer long-term profiles.

Exemplary oral care products suitable for use in the aforementioned methods may comprise a base including but not limited to the following materials: polysaccharides and polysaccharide derivatives, proteins, lipids, in an orally acceptable vehicle including humectants, sweetening agents, flavoring agents, buffering agents, water and/or preservatives. The application of the base alone inhibits plaque accumulation, prevent tooth decay and periodontal diseases. In another embodiment, the ingestible oral care compositions further comprise one or more pH neutralizing agents e.g., inorganic salts, which may, for example, neutralize acids on site and/or further prevent tooth decay. In another embodiment, the oral compositions further comprise one or more of oral health promoting actives including but not limited to antioxidants, anti-inflammatory agents, anti microbial agents, nutrients, sensory agents, plant extracts, essential oils and etc. for management of local pain, sensitivity, gingival inflammation, swollen gums, bleeding, gum recession, malodor and etc. or potentially control of systematic or respiratory health concerns. The oral compositions, in another embodiment carry drug actives in the base for targeted topical delivery to the base of the teeth and/or the periodontal pockets as a safe and efficacious treatment option for gum diseases.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should rather be given the broadest interpretation consistent with the description as a whole.

### BRIEF DESCRIPTION OF THE DRAWING

FIG. 1 is a plot showing a comparison between stained bacteria count for samples taken from an oral cavity of subjects in which an ingestible oral care composition according to an example of the present disclosure had been applied (left columns) in comparison to control (right columns) for, from left to right, samples taken at times of 0, 15, 30, 60, 90 and 120 minutes.

### DETAILED DESCRIPTION

### I. Definitions

Unless otherwise indicated, the definitions and embodiments described in this and other sections are intended to be applicable to all embodiments and aspects of the disclosure herein described for which they would be understood to be suitable by a person skilled in the art.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process/method steps. As used herein, the word “consisting” and its derivatives, are intended to be close ended terms that specify the presence of stated features, elements, components, groups, integers, and/or steps, and also exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this disclosure, the singular forms “a”, “an” and “the” include plural references unless the content clearly dictates otherwise.

The term “and/or” as used herein means that the listed items are present, or used, individually or in combination. In effect, this term means that “at least one of” or “one or more” of the listed items is used or present.

By “ingestible” it is meant, for example, that the components of the product/composition, especially the components in the base are considered suitable for consumption by a subject in the levels used in the products/compositions of the disclosure, for example, they may be Generally Recognized As Safe (GRAS) materials by the United States Food and Drug Administration (FDA) and/or food grade, whereas the additional actives may, for example, be GRAS materials, food grade materials and/or or otherwise used in a level that is suitable for daily oral consumption. It will be appreciated by a person skilled in the art that in embodiments wherein the active comprises a pharmaceutically active compound, such an active is desirably present in a form that is, for example, compatible with the treatment of subjects via delivery through an ingestible oral care composition and is for administration/use in a subject in an amount effective, at dosages and for periods of time necessary to achieve a desired result. Thus, the products/ingestible oral care compositions of the disclosure are suitable to be left in the oral cavity, for example, after daily application, or multiple applications within a day, or with prescription depending on the embodiment. The ingestible oral care compositions of the disclosure therefore may, for example, advantageously work as a leave in oral care product for a prolonged period of time on tooth and gum surfaces to destabilize plaque, prevent regrowth of plaque, and/or reduce plaque non-invasively. Alternatively, in some embodiments, the ingestible oral care composition is for temporary therapeutic use.

The term “non-invasively” and the like as used herein means, for example, without application of significant mechanical force, brushing and/or antimicrobial chemicals.

By “prolonged time” is meant the time after the product is applied on tooth and gum surfaces, the product stays on these surfaces for 10 minutes or longer. The length of the residence time is affected by the oral activity after application. In an embodiment, longer residence time is achieved by applying the product using a soft brush all over the tooth and gum surfaces after cleaning the teeth with toothpaste and/or floss before going to bed at night.

By “appealing mouth feel and convenient application method” is meant that the product consistency and texture is of a semi-solid nature, similar to commercial toothpastes. The product can be packaged in similar way as toothpastes or differently. The product can also be brushed on the gum and tooth surfaces with tooth brushes, preferably with gentle motion by soft toothbrushes to cover all the surface areas. The product thus applied is a gel by nature and subsequently dissolves and disperses into the oral cavity with no solid residues left in mouth over time, but preferably a pleasant and refreshing flavor as embedded in the product. The product applied in this manner can be easily added to current daily oral care routines after cleaning with toothpaste and floss, as a supplemental leave in tooth and gum conditioning step, which makes use of functional oral care actives in a safer and more efficacious way than when they are used in toothpastes or mouth rinses, whereas the aforementioned actives are spitted or rinsed off from the mouth with only a short contact time.

The term “oral surface” as used herein includes any surface in the mouth suitable for application of an ingestible oral care composition thereon such as suitable mucosal surfaces (e.g., gingiva), natural teeth, artificial teeth (e.g., implants) and teeth with dental restorations thereon (e.g., crowns or bridges) as well as combinations thereof.

### II. Ingestible Oral Care Compositions and Uses Thereof

The present disclosure includes oral care products comprising a base that is made up of the following: polysaccharides and/or polysaccharide derivatives, proteins, lipids, in an orally acceptable vehicle including humectants, sweetening agents, flavoring agents, buffering agents, water and/or preservatives. In the aforementioned oral care products, carefully selected polysaccharides and/or polysaccharide derivatives, proteins, and lipids and optionally inorganic salts which are either part of the natural biofilm composition or specific replacements that are highly biocompatible are combined, for example, with humectants, sweetening agents, flavoring agents and water to prepare the aforementioned product which also adheres to tooth and gum surfaces, but in a consistency and texture that can be easily applied on the local surfaces and stay there for a certain period of time before fully dispersing into the oral cavity. Once the aforementioned product is applied to the local surfaces after tooth cleaning procedures, the residence of the product on the local surfaces destabilizes residue plaque, forms a physical barrier to delay the otherwise reoccurrence of biofilm formation on the tooth surfaces and further reduce the chances of plaque formation and accumulation on tooth surfaces. Optionally, the inorganic salts are included in the product to neutralize acids on site, which further prevents tooth decay and reduces gingival inflammation and related local bleeding and/or swelling. Optionally, actives are included for local delivery.

The present disclosure also includes an ingestible oral care composition comprising: polysaccharides and/or derivatives thereof; protein; lipids; an inorganic salt pH neutralizing agent; a humectant; a sweetener; a flavor; water; and optionally an active. The present disclosure also includes an ingestible oral care composition comprising: protein; lipids; polysaccharides and/or derivatives thereof; water; and optionally an inorganic salt pH neutralizing agent; a humectant; a sweetener; a flavor; and/or an active.

The present disclosure also includes an ingestible oral care composition, comprising: protein; lipids; polysaccharides and/or derivatives thereof; and optionally an active, in a water-based orally acceptable vehicle.

The protein is any suitable protein or combinations thereof and is present in any suitable amount. In an embodiment, the protein is present in an amount of from about 0.05 wt % to about 10 wt %, based on the total weight of the ingestible oral care composition. In another embodiment, the protein is present in an amount of from about 0.1 wt % to about 5 wt %, based on the total weight of the ingestible oral care composition. The said proteins in the aforementioned products include but are not limited to collagen, gelatin, soy protein, pea protein, especially proline rich proteins for adherence to tooth surfaces. Accordingly, a person skilled in the art would readily appreciate that the term “protein” as used herein also includes suitable substances derived from protein such as but not limited to gelatin and/or hydrolyzed collagen as well as substances comprising suitable mixtures of proteins such as but not limited to soy protein, pea protein, rice protein and/or whey protein. In an embodiment, the protein is a proline-rich protein. In an embodiment, the protein is gelatin, hydrolyzed collagen, pea protein, soy protein, rice protein, whey protein or combinations thereof. In another embodiment, the protein is selected from collagen, gelatin, soy protein, pea protein and combinations thereof. In an embodiment, the protein is a combination of hydrolyzed collagen and gelatin. Gelatin is a preferred choice for its high biocompatibility and its potential to prevent gum recession and boost gum healing, regrowth and attachment to the tooth. In an embodiment, the protein is gelatin. In a further embodiment, the gelatin is present in an amount of 0.1 wt % to about 5 wt %, based on the total weight of the ingestible oral care composition.

The polysaccharides and/or derivatives thereof are any suitable polysaccharide, polysaccharide derivative or combinations thereof and are present in any suitable amount. In an embodiment, the polysaccharides and/or derivatives thereof are present in an amount of from about 0.3 wt % to about 42 wt %, based on the total weight of the ingestible oral care composition. In an embodiment, the polysaccharides and/or derivatives thereof are present in an amount of about 1.6% to about 27 wt %, based on the total weight of the ingestible oral care composition. The said polysaccharides and/or polysaccharide derivatives in the aforementioned products include but are not limited to plant based materials such as cellulose and cellulose derivatives, pectin, acacia gum, gum arabic, inulin, guar gum, tara gum, and locust bean gum; animal based materials such as hyaluronic acid, sodium hyaluronate, chitin, chitosan, and heparin; microbe based materials such as pullulan, xanthan gum, gellan gum, and succinoglycan; and algae based materials such as alginate, agar, carrageenan, fucoidan and etc. In an embodiment, the polysaccharides and/or derivatives thereof are selected from plant-based materials, animal-based materials, microbe-based materials, algae-based materials and combinations thereof. In another embodiment, the polysaccharides and/or derivatives thereof are selected from cellulose, cellulose derivatives, pectin, acacia gum, arabinogalactan-containing gum, gum arabic, inulin, guar gum, tara gum, locust bean gum, hyaluronic acid, sodium hyaluronate, chitin, chitosan, heparin, pullulan, xanthan gum, gellan gum, succinoglycan, algin, alginate, agar, carrageenan, fucoidan and combinations thereof. One or more of the above materials are selected or combined to contribute to the aforementioned properties, among which one of the preferred choices is a combination of pectin, gum arabic, guar gum, xanthan gum and alginate. Accordingly, in an embodiment, the polysaccharides and/or derivatives thereof are a combination of gum arabic, pectin, guar gum, xanthan gum and sodium alginate. In another embodiment, the polysaccharides and/or derivatives thereof are a combination of arabinogalactan-containing gums, guar gum, xanthan gum and sodium alginate. The arabinogalactan-containing gums are any suitable arabinogalactan-containing gum or combinations thereof. In an embodiment, the arabinogalactan-containing gum is larch arabinogalactan, gum ghatti, gum arabic or combinations thereof. In another embodiment, the polysaccharides and/or derivatives thereof are a combination of gum arabic, guar gum, xanthan gum and sodium alginate. In an embodiment, the arabinogalactan-containing gums are present in an amount of from about 0.1 wt % to about 30 wt %; the guar gum is present in an amount of from about 0.05 wt % to about 1 wt %; the xanthan gum is present in an amount of from about 0.05 wt % to about 1 wt %; and the sodium alginate is present in an amount of from about 0.1 wt % to about 10 wt %, based on the total weight of the ingestible oral care composition. In an embodiment, the arabinogalactan-containing gum is gum arabic and is present in an amount of from about 1 wt % to about 20 wt %; the guar gum is present in an amount of from about 0.05 wt % to about 0.5 wt %; the xanthan gum is present in an amount of from about 0.05 wt % to about 0.5 wt %; and the sodium alginate is present in an amount of from about 0.5 wt % to about 6 wt %, based on the total weight of the ingestible oral care composition.

The lipids are any suitable lipids or combinations thereof and are present in any suitable amount. In an embodiment, the lipids are present in an amount of from about 0.2 wt % to about 27 wt %, based on the total weight of the ingestible oral care composition. In another embodiment, the lipids are present in an amount of from about 1 wt % to about 10 wt %, based on the total weight of the ingestible oral care composition. The said lipids in the aforementioned products include but are not limited to medium chain triglycerides, grape seed oil, flax seed oil, avocado oil, coconut oils, sunflower oils, olive oils, sesame oils and etc. The lipids may provide anti oxidant and/or anti-inflammatory effects. One of the preferred choices is caprylic/capric triglyceride. Accordingly, a person skilled in the art would readily appreciate that the term “lipids” as used herein also includes suitable combinations of lipids as well as suitable substances derived from and/or prepared from lipids such as but not limited to caprylic/capric triglyceride. In an embodiment, the lipids are medium chain triglycerides, grape seed oil, flax seed oil, avocado oil, coconut oils, palm oil, sunflower oils, olive oils, sesame oils or combinations thereof. In another embodiment, the lipids are caprylic/capric triglyceride. In another embodiment, the caprylic/capric triglyceride is present in an amount of from about 1 wt % to about 10 wt %, based on the total weight of the ingestible oral care composition.

In an embodiment, the ingestible oral care composition further comprises a pH neutralizing agent. In an embodiment, the pH neutralizing agent is an inorganic salt pH neutralizing agent. The inorganic salt is any suitable inorganic salt or combinations thereof capable of acting as a pH neutralizing agent in an ingestible oral care composition and is present in any suitable amount. For example, in the ingestible oral care compositions of the disclosure, the pH neutralizing agent is advantageously capable of neutralizing acids on site for an extended period of time which may, for example, prevent caries and/or tooth decay. In an embodiment, the pH neutralizing agent is present in an amount of from about 0.2 wt % to about 3 wt %, based on the total weight of the ingestible oral care composition. The said inorganic salts in the aforementioned products include but are not limited to: dicalcium phosphate dihydrate, dicalcium phosphate anhydrous, and tricalcium phosphate. Accordingly, in an embodiment, the pH neutralizing agent is dicalcium phosphate anhydrous, dicalcium phosphate dihydrate, tricalcium phosphate or combinations thereof. The said inorganic salts are fine powders that are not abrasive and with limited solubility in water. Once applied, the salt powders embedded in the product reside on the tooth and gum surfaces, continuously neutralize local acids and maintain neutral pH level to prevent erosion on tooth surfaces and tooth decay, inhibit plaque accumulation, and prevent gum diseases. Combinations of the above salts in specific ratios may also provide pH adjusting function for the overall pH of the aforementioned products to be between 6.5-7.5. One of the preferred choices is a combination of dicalcium phosphate anhydrous and tricalcium phosphate with fine particle sizes. Accordingly, in an embodiment, the ingestible oral care composition comprises a combination of dicalcium phosphate anhydrous and tricalcium phosphate.

It will be appreciated by a person skilled in the art that the water-based orally acceptable vehicle may, in addition to water, comprise ingredients selected from one or more of, e.g., a buffering agent, a humectant, a sweetener, a flavor and a preservative. The water-based orally-acceptable vehicle can be present in any suitable amount to achieve the desired product, the determination of which can be made by a person skilled in the art. For example, it will be appreciated by a person skilled in the art that the ingestible oral care compositions of the disclosure are not in the form of a film but rather are in the form of a semi-solid such as a gel.

The buffering agent is any suitable buffering agent or combinations thereof and is present in any suitable amount. In an embodiment, the buffering agent is trisodium phosphate, sodium citrate, citric acid and sodium hydroxide or combinations thereof. In another embodiment, the buffering agent is a combination of sodium citrate, citric acid and sodium hydroxide. In contrast to the pH neutralizing agent which is used, for example, to have a neutralizing effect when applied an oral surface of the subject, the buffering agent and amounts thereof are selected to have an effect on the product/ingestible oral care compositions of the disclosure. In an embodiment, the buffering agent is present in an amount of about 0.1 wt % to about 1 wt %, based on the total weight of the ingestible oral care composition.

The humectant is any suitable humectant or combinations thereof and is present in any suitable amount. The said humectants in the aforementioned products include but are not limited to glycerin, propylene glycol, butylene glycol, propanediol, pentylene glycol, and other organic ones such as aloe vera. The humectants hold water in the aforementioned product, contribute to the formation of the semi solid structure and prevent microbial growth in the aforementioned product. One of the preferred choices is a combination of glycerin with aloe vera for aloe vera's soothing property. Accordingly, in an embodiment, the humectant is a combination of glycerin and aloe vera. In another embodiment, the humectant comprises, consists essentially of or consists of glycerin. In a further embodiment, the humectant comprises glycerin.

The sweetener is any suitable sweetener or combinations thereof and is present in any suitable amount. The said sweeteners in the aforementioned products include but are not limited to sugar alcohols, such as xylitol, erythritol, sorbitol, among which the low/zero calorie ones like erythritol and xylitol are preferred for their well-established benefits used in oral care products; and other sugar substitutes, such as pure stevia, monk fruit extract, Acesulfame potassium, Advantame, Aspartame, Neotame, Saccharin and Sucralose. In an embodiment, the sweetener is selected from sugar alcohols, other sugar substitutes and combinations thereof. In another embodiment, the sweetener is selected from xylitol, erythritol, sorbitol, pure stevia, monk fruit extract, Acesulfame potassium, Advantame, Aspartame, Neotame, Saccharin and Sucralose. Erythritol and/or a combination of erythritol and xylitol is a preferred choice for the product. In an embodiment, the sweetener is erythritol and/or a combination of erythritol and xylitol. In another embodiment, the sweetener comprises, consists essentially of or consists of xylitol. In a further embodiment, the sweetener comprises xylitol.

The flavor is any suitable flavor or combinations thereof and is present in any suitable amount. The said flavors in the aforementioned product include but are not limited to peppermint and spearmint. The preferred flavor is the mint flavor including one or both of peppermint and spearmint. Accordingly, in an embodiment, the flavor is mint flavor. In another embodiment, the flavor is peppermint, spearmint or a combination thereof.

Wherein the said components in the aforementioned product, when combined properly with water not only provide the said base with the forementioned properties, but also a dental friendly microbiome environment, when applied, supports the diversity of the microbiome in oral cavity and inhibits the overgrowth of acidogenic and/or pathogenic microbiomes. In an embodiment, the proper combination of the aforementioned preferred choices of each component includes a specific ratio of each ingredient, an order of addition with specific mixing conditions and temperatures, of which when optimized can achieve the described physical and chemical properties wherein aforementioned. However, a person skilled in the art will appreciate that the products/compositions can readily be made using methods which are common in the oral product area and could readily select a suitable method.

In some embodiments, the products/compositions comprise the active. The active may therefore be deliverable to the oral cavity through the ingestible oral care compositions of the disclosure. The active is any suitable active or combinations thereof and is present in any suitable amount, which may depend, for example, on the identity of the active or combination thereof. Actives with specific health promoting functions and compatible physical and chemical properties such as pH, solubility and stability with the base can be embedded in ways straightforward to those skilled in the art. The aforementioned actives can advantageously be used with relatively lower concentrations but generate potentially higher efficacy and better long-term safety profiles due to the prolonged time on site to exert functions locally on targets on tooth and gum surfaces to reduce plaque, control gingivitis, prevent periodontitis and prevent tooth decay. The said aforementioned actives include but are not limited to, antioxidants, anti-inflammatory agents, anti microbial agents, nutrients such as vitamins, sensory agents, plant extracts, essential oils and etc. In another embodiment, the active is selected from antioxidants, anti-inflammatory agents, antimicrobial agents, nutrients (such as vitamins), sensory agents, plant extracts, essential oils and combinations thereof. In another embodiment, the active comprises a vitamin. The vitamin can be any suitable vitamin or combinations thereof. In another embodiment, the vitamin is a combination of vitamin C and vitamin B12. In a further embodiment, the vitamin C is present in an amount of about 0.1 wt % to about 2 wt % and the vitamin B12 is present in an amount of about 0.001 wt % to about 0.05 wt %. In another embodiment, the active comprises plant extracts. The plant extracts can be any plant extracts or combinations thereof suitable for use in an ingestible oral care composition. In another embodiment, the active comprises essential oils. The essential oils can be any essential oils or combinations thereof suitable for use in an ingestible oral care composition.

Wherein the products with the aforementioned actives, can be applied as described above as an overall conditioning complements to the tooth cleaning procedures, it can also be applied on independently or with other oral procedures to specific spots on the tooth and gum surfaces to relieve certain tooth and/or gum concerns, such as swollen gum, pain, bleeding, sensitivity, malodor, periodontal inflammations and etc. whenever necessary, with or without the help of a clean dental floss to push the product further into the base of the teeth and/or into the periodontal pocket. In some embodiments, oral care products/compositions that are safe to be ingested and suitable for long term daily use in the oral cavity with appealing mouth feel and convenient application method are provided. Unlike toothpaste and oral rinse, which needs to be spitted out or rinsed off afterwards, the product is designed to be left in the oral cavity on tooth and gum surfaces after application. The product slowly disperses into the local environment, thus giving its ingredients a prolonged time window to perform locally on tooth and gum surfaces. The product is not for cleaning purpose, but for providing better maintenance and protection through prolonged local existence in the oral cavity, especially on and/or around tooth and gum surfaces to prevent tooth decay and reduce periodontal diseases. The product may comprise a semisolid base and additional actives that attribute to specific functions locally on and/or around tooth and gum surfaces, and possible systemic benefits or benefits to respiratory system after ingestion.

It is also a feature included herein to use the aforementioned base as a drug delivery vehicle to deliver pharmaceutical actives specifically to periodontal pockets and surrounding areas with/without the aid of curve-tipped dental syringes or other fine tipped dental tools. Accordingly, in an embodiment, the active comprises a pharmaceutically active compound. In another embodiment, the active comprises a pharmaceutically active compound, a vitamin, a plant extract, an essential oil or combinations thereof. In a further embodiment, the active comprises a pharmaceutically active compound, and optionally a vitamin, a plant extract, an essential oil or combinations thereof. Because of the high biocompatibility of the base, the aforementioned drug delivery system can also be applied to similar targeted areas for implants, teeth with crowns or bridges, or other natural or artificial teeth after operations to control pain and inflammation with minimal side effects. The pharmaceutical actives aforementioned are preferably compatible with the base in physical and chemical properties including but not limited to pH, solubility and stability. In another feature included herein, the aforementioned product includes base materials and functional actives that promote microbiome balances and/or overall health not only for oral cavity but also for respiratory and/or systemic systems with regular use. Examples of the aforementioned base materials include but are not limited to prebiotics such as pectin and/or inulin; examples of the aforementioned actives include but are not limited to plant extracts such as green tea extract and/or licorice extract.

The present disclosure also includes a use of an ingestible oral care composition as described herein for controlling gum inflammation in a subject. The present disclosure also includes a use of an ingestible oral care composition as described herein for preparation of a medicament for controlling gum inflammation in a subject. The present disclosure also includes an ingestible oral care composition as described herein for use in controlling gum inflammation in a subject. The present disclosure also includes a method of controlling gum inflammation in a subject, comprising applying an ingestible oral care composition as described herein to an oral surface of the subject. In an embodiment, the oral surface comprises a surface of the gingiva or a portion thereof.

The present disclosure also includes a use of an ingestible oral care composition as described herein, which comprises an active, for controlling respiratory diseases through mouth and throat in a subject. The present disclosure also includes a use of an ingestible oral care composition as described herein, which comprises an active, for preparation of a medicament for controlling respiratory diseases through mouth and throat in a subject. The present disclosure also includes an ingestible oral care composition as described herein, which comprises an active, for use in controlling respiratory diseases through mouth and throat in a subject. The present disclosure also includes a method of controlling respiratory diseases through mouth and throat in a subject in need thereof, comprising applying an ingestible oral care composition as described herein, comprising an active, to an oral surface of the subject.

The present disclosure also includes a use of an ingestible oral care composition as described herein for preventing systemic diseases related to periodontal inflammation in a subject. The present disclosure also includes a use of an ingestible oral care composition as described herein for preparation of a medicament for preventing systemic diseases related to periodontal inflammation in a subject. The present disclosure also includes an ingestible oral care composition as described herein for use in preventing systemic diseases related to periodontal inflammation in a subject. The present disclosure also includes a method of preventing systemic diseases related to periodontal inflammation in a subject, comprising applying an ingestible oral care composition as described herein to an oral surface of the subject.

The present disclosure also includes a use of an ingestible oral care composition as described herein for non-invasively destabilizing plaque, preventing regrowth of plaque and/or reducing plaque on an oral surface of a subject. The present disclosure also includes an ingestible oral care composition as described herein for use in non-invasively destabilizing plaque, preventing regrowth of plaque and/or reducing plaque on an oral surface of a subject. The present disclosure also includes a method of non-invasively destabilizing plaque, preventing regrowth of plaque and/or reducing plaque on an oral surface of a subject, comprising applying an ingestible oral care composition as described herein to the oral surface of the subject.

The present disclosure also includes a use of an ingestible oral care composition as described herein, which comprises an active, for delivering the active to a subject in need thereof. The present disclosure also includes a use of an ingestible oral care composition as described herein, which comprises an active, for preparation of a medicament for delivering the active to a subject in need thereof. The present disclosure also includes an ingestible oral care composition as described herein, which comprises an active, for use in delivering the active to a subject in need thereof. The present disclosure also includes a method of delivering an active to a subject in need thereof, comprising applying an ingestible oral care composition as described herein, comprising an active, to an oral surface of the subject.

In an embodiment, the subject is a human.

The following are non-limiting examples of the present disclosure:

### EXAMPLES

### Example 1

The ingestible oral care compositions of the disclosure can be made using methods which are common in the oral product area. The components of an exemplary oral care composition are shown in Table 1. In an exemplary procedure for preparation of the ingestible oral care composition, gelatin and the polysaccharides are first dispersed and hydrated in the orally acceptable vehicle. The medium chain triglyceride surprisingly functions as a crosslinker between the protein and the polysaccharides and increases the viscosity of the biopolymer complex base. Once the biopolymer complex base is formed, the rest of the ingredients for pH neutralizing, flavor and the actives are introduced into the base slowly to avoid trapping air. The buffering agents, sweetener, flavor and humectant such as glycerin are introduced into water to form the orally acceptable vehicle.

TABLE 1

| | Component | % (w/w) |
|---|---|---|
| Natural | gelatin | 0.50 |
| Biopolymer | medium chain triglyceride (caprylic/ | 3.00 |
| Complex base | capric triglyceride) | |
| | gum arabic | 2.00 |
| | sodium alginate | 2.00 |
| | xanthan gum | 0.30 |
| | guar gum | 0.30 |
| | pH neutralizing agent | 1.0 |
| Orally | sodium citrate, citric acid and sodium | 0.5 |
| acceptable | hydroxide | |
| vehicle | xylitol | 2.00 |
| | flavor | 0.15 |
| | water and glycerin | 87.24 |
| Actives | vitamin C | 1.00 |
| | vitamin B12 | 0.01 |
| | | 100 |

Gelatin was included for its biocompatibility and potential as a proline rich protein to facilitate adhesion to the teeth surface. The initial examples were prepared with pectin, gum arabic, guar gum, xanthan gum and alginate. This mixture of polysaccharides was useful to prepare ingestible oral care compositions of the disclosure. However, variation of pectin sources and inconsistencies in the final mixture led to a focus on the other four natural gums.

A challenge for the preparation of ingestible oral care products is retention of the product locally using ingestible ingredients. A mixture of gelatin and the polysaccharides as illustrated in Table 1 showed adhesion to the tooth surface. However, it only stayed for a very short amount of time (less than 15 minutes). Surprisingly, when medium chain triglyceride was added to the gelatin and polysaccharides base, the viscosity increased, the consistency of the base was improved and the retention on the surface of the teeth increased to close to 30 minutes. However, it was also found that the amount of medium chain triglyceride that can be well incorporated into the gelatin and polysaccharides base was proportional to the other biopolymers. The amount listed in Table 1 produced a gel base that is applicable as an oral care product in a squeezable container and similar proportions are expected to behave similarly.

Another challenge of ingestible oral care products is the potential of plaque regrowth (see, for example: Rubido et al., In vivo antiplaque effect of three edible toothpastes Med Oral Patol Oral Cir Bucal. 2014 Jan. 1; 19(1):e88-92). The effect of individual gum solutions was tested on beta-glucan which is a similar polysaccharide to the one in EPS. Among them, surprisingly, beta-glucan in a gel form slowly disappeared in size in the gum solutions with the speed in the following order: gum arabic>alginate>guar gum>xanthan gum. Other arabinogalactan-containing gums similar to gum arabic, which are highly branched polysaccharides with 1-arabinose side chains, were thus also tested. Larch arabinogalactan and gum ghatti showed a similar effect on beta-glucan gel in the solution to gum arabic.

After applying the product of Table 1 on beta-glucan gel, similar effects were observed as the beta-glucan gel starting to reduce in size. Since beta-glucan is the major component of biofilm, this indicates a potential interaction between the product and the biofilm polysaccharides which could, for example, lead to the destabilization and deterioration of the biofilm and prevent further plaque growth.

The product of Table 1 includes gelatin, caprylic/capric triglyceride, and a polysaccharide combination of sodium alginate, gum arabic, xanthan gum and guar gum. The protein, lipids and polysaccharides in the EPM contribute to the biofilm as energy sources, structural components and communication liaisons. All these molecules are involved in the bacteria attachment to the biofilm and later interaction in the biofilm. Unlike previous products where polymers or polysaccharides are used as the viscous carriers for attachment to the dental surface, the polysaccharide combination in this product is not viscous enough and can not attach to the dental surface by itself. While not wishing to be limited by theory, these polysaccharides especially sodium alginate and gum arabic seem to have antagonism with glucans which are the major component of the EPM polysaccharides. Xanthan gum and guar gum are both advantageously used at a minimum level to facilitate the final viscoelastic property of the product, but not used as viscosity builders. Gelatin as the most biocompatible protein with different hydrophilic (anionic, cationic) and hydrophobic parts on its amino acid chain, interacts with the lipids and polysaccharides as the key structural backbone and helps to attach these components to the dental biofilm. The medium chain triglycerides interact with gelatin and the polysaccharide combination as well, which surprisingly increased the viscosity of the complex, while not wishing to be limited by theory, indicating a possible weak crosslinking effect on the complex.

### Example 2

Before taking samples from oral cavity, subjects were asked to brush teeth with regular toothpaste for 3 minutes. After brushing teeth, subjects applied the oral healthcare gel product of Table 1 on the tooth surface at gingival edge, and used a soft toothbrush to make it even and into the gingival groove as much as possible. Samples were taken with applicators at 0, 15, 30, 60, 90 and 120 mins. The samples were placed into 1.5 mL vials and each vial contained 200 μL methylene blue (a simple stain) to mix the sample with. Then, 10 μL of the mixed solution was taken into a hemocytometer to count the number of bacteria. Based on the counted number, the number of bacteria of each sample was calculated.

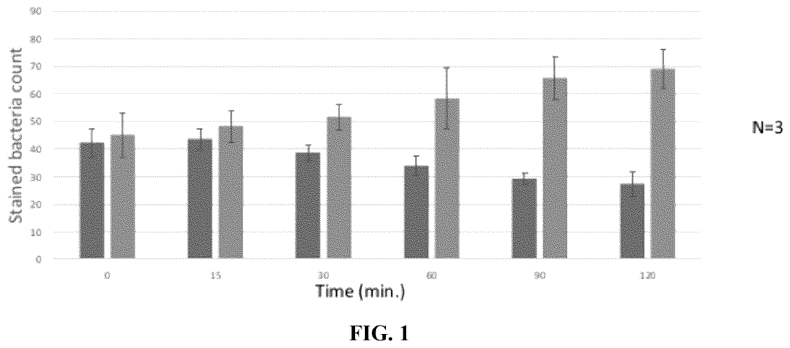

A reduction of bacteria was observed after applying the gel (FIG. 1; n=3).

### Example 3

A 50-year-old healthy female subject applied the oral healthcare gel product of Table 1 with a soft bristle brush after brushing before going to bed each night for two weeks. After the two weeks of continuous use, an indication of control of gum recession was observed as well as a reduction of gum inflammation.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

1. An ingestible oral care composition, comprising:
protein;
lipids;
polysaccharides and/or derivatives thereof; and
optionally an active,
in a water-based orally acceptable vehicle.

2. The ingestible oral care composition of claim 1, further comprising a pH neutralizing agent.

3. The ingestible oral care composition of claim 1 or 2, wherein the polysaccharides and/or derivatives thereof are a combination of arabinogalactan-containing gums, guar gum, xanthan gum and sodium alginate.

4. The ingestible oral care composition of claim 1 or 2, wherein the polysaccharides and/or derivatives thereof are a combination of gum arabic, pectin, guar gum, xanthan gum and sodium alginate.

5. The ingestible oral care composition of claim 1 or 2, wherein the polysaccharides and/or derivatives thereof are a combination of gum arabic, guar gum, xanthan gum and sodium alginate.

6. The ingestible oral care composition of claim 3, wherein the arabinogalactan-containing gums are present in an amount of from about 0.1 wt % to about 30 wt %; the guar gum is present in an amount of from about 0.05 wt % to about 1 wt %; the xanthan gum is present in an amount of from about 0.05 wt % to about 1 wt %; and the sodium alginate is present in an amount of from about 0.1 wt % to about 10 wt %, based on the total weight of the ingestible oral care composition.

7. The ingestible oral care composition of claim 6, wherein the arabinogalactan-containing gum is gum arabic and is present in an amount of from about 1 wt % to about 20 wt %; the guar gum is present in an amount of from about 0.05 wt % to about 0.5 wt %; the xanthan gum is present in an amount of from about 0.05 wt % to about 0.5 wt %; and the sodium alginate is present in an amount of from about 0.5 wt % to about 6 wt %, based on the total weight of the ingestible oral care composition.

8. The ingestible oral care composition of any one of claims 1 to 7, wherein the protein is gelatin, hydrolyzed collagen, pea protein, soy protein, rice protein, whey protein or combinations thereof.

9. The ingestible oral care composition of any one of claims 1 to 8, wherein the protein is a combination of hydrolyzed collagen and gelatin.

10. The ingestible oral care composition of claim 9, wherein the protein is present in an amount of from about 0.05 wt % to about 10 wt %, based on the total weight of the ingestible oral care composition.

11. The ingestible oral care composition of any one of claims 1 to 8, wherein the protein is gelatin.

12. The ingestible oral care composition of claim 11, wherein the gelatin is present in an amount of from about 0.1 wt % to about 5 wt %, based on the total weight of the ingestible oral care composition.

13. The ingestible oral care composition of any one of claims 1 to 12, wherein the lipids are medium chain triglycerides, grape seed oil, flax seed oil, avocado oil, coconut oils, palm oil, sunflower oils, olive oils, sesame oils or combinations thereof.

14. The ingestible oral care composition of claim 13, wherein the lipids are present in an amount of from about 0.2 wt % to about 27 wt %, based on the total weight of the ingestible oral care composition.

15. The ingestible oral care composition of any one of claims 1 to 12, wherein the lipids are caprylic/capric triglyceride.

16. The ingestible oral care composition of claim 15, wherein the caprylic/capric triglyceride is present in an amount of from about 1 wt % to about 10 wt %, based on the total weight of the ingestible oral care composition.

17. The ingestible oral care composition of any one of claims 2 to 16, wherein the pH neutralizing agent is dicalcium phosphate anhydrous, dicalcium phosphate dihydrate, tricalcium phosphate or combinations thereof.

18. The ingestible oral care composition of claim 17, wherein the pH neutralizing agent is present in an amount of from about 0.2 wt % to about 3 wt %, based on the total weight of the ingestible oral care composition.

19. The ingestible oral care composition of any one of claims 1 to 18, wherein the ingestible oral care composition comprises the active.

20. The ingestible oral care composition of claim 19, wherein the active comprises a vitamin.

21. The ingestible oral care composition of claim 20, wherein the vitamin is a combination of vitamin C and vitamin B12.

22. The ingestible oral care composition of claim 19, wherein the active comprises plant extracts.

23. The ingestible oral care composition of claim 19, wherein the active comprises essential oils.

24. The ingestible oral care composition of claim 19, wherein the active comprises a pharmaceutically active compound, a vitamin, a plant extract, an essential oil or combinations thereof.

25. The ingestible oral care composition of claim 19, wherein the active comprises a pharmaceutically active compound.

26. A use of the ingestible oral care composition of any one of claims 1 to 25, for controlling gum inflammation in a subject.

27. A use of the ingestible oral care composition of claim 24 or claim 25, for controlling respiratory diseases through mouth and throat in a subject.

28. A use of the ingestible oral care composition of any one of claims 1 to 25, for preventing systemic diseases related to periodontal inflammation in a subject.

29. A use of the ingestible oral care composition of any one of claims 1 to 25 for non-invasively destabilizing plaque, preventing regrowth of plaque and/or reducing plaque on an oral surface of a subject.

30. A use of the ingestible oral care composition of any one of claims 19 to 25, for delivering the active to a subject in need thereof.
